(19) United States
(12) Patent Application Publication
(10) Pub. No.: US 2004/0086566 A1
(43) Pub. Date: May 6, 2004

(54) Waxy matrix dosage forms

(75) Inventor: Xiaoying Zhang, Morris Plains, NJ (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: ALPHARMA, INC., Fort Lee, NJ

(21) Appl. No.: 10/287,410

(22) Filed: Nov. 4, 2002

(51) Int. Cl. A61K 9/24; A61K 9/14

(52) U.S. Cl. 424/484; 424/471

(57) ABSTRACT

In a preferred embodiment of the invention, a solid dosage form is provided comprising a matrix, wherein the matrix comprises (a) a pharmaceutically effective amount of metformin or a pharmaceutically acceptable salt thereof and (b) a waxy matrix material. The invention also provides a method of making a solid dosage form, the method comprising: (a) hot melting a waxy material to form a melt, (b) granulating metformin or a pharmaceutically acceptable salt thereof with the melt to form a granulate; (c) milling the granulate; and (d) compressing granulate to form a matrix.

### FIELD OF THE INVENTION

The present invention relates to waxy matrix dosage forms. In a preferred embodiment, the invention relates to a solid dosage form comprising a high loading dose of a highly water soluble active agent in a waxy matrix. Preferred active agents include metformin and pharmaceutically acceptable salts thereof.

### BACKGROUND OF THE INVENTION

Metformin and its salts, particularly metformin hydrochloride, have been employed as a pharmaceutically active agent in the treatment of diabetes. The dosage required of this drug is high, especially in extended release dosage forms. For example, current dosage forms contain 500, 850, or 1000 mg of metformin agent product (e.g. GLUCOPHAGE XR® (Bristol Myers Squibb)) or 125, 250, or 500 mg of metformin hydrochloride combined with other active agents (e.g. GLUCOVANCE® (Bristol Myers Squibb)). When a high dosage of metformin is combined with excipients, the resulting dosage form (e.g., tablets, capsules, etc.) is considerably larger in size than is desirable. Also, the dosage form can be undesirably large when metformin is combined with other active agents, especially other high dose active agents. The large size of these dosage forms are frequently difficult for the patient to swallow, particularly for older patients, which make up a large part of the diabetic population. Further, the large size of the dosage form may increase the risk of choking upon oral administration.

Manufacturing problems associated with high dosage forms of an active agent are known, such as suitable compression and moisture, especially in the manufacture of tablets. For example, metformin requires carefully controlled amounts of water to be present during tablet compression to control capping. Capping denotes the detachment of layers of compressed mass during the pressing or shortly thereafter. Capping can be caused by any number of problems, including inadequate binding agent action, inadequate or excessive moisture content of the granulate, unsuitable crystal forms, strongly aerophilic substances, excessive porosity, excessive proportion of powder, excessive interparticulate binding between the granulate particles and unsuitable granulate forms. Machine factors may also lead to capping, including excessive pressing force, badly applied or worn tools, excessive pressing rages and poor deaeration of the matrix (fixed pressure). However, in the case of high dose active agents, such as metformin, the usual measures are often inadequate to suitably control the capping of the tableting mass.

Therefore, there exists a need to control the capping of a tableting mass comprising a high dose amount of an active agent(s). There also exists a need for a dosage form comprising a high dose amount of the active agent(s) that has a smaller size than conventional dosage forms containing substantially the same dose amount of the active agent(s). These and other advantages of the invention, as well as additional inventive features, will be apparent from the description of the invention provided herein.

### BRIEF SUMMARY OF THE INVENTION

In a preferred embodiment of the invention, a solid dosage form is provided comprising a matrix comprising: (a) a pharmaceutically effective amount of metformin or a pharmaceutically acceptable salt thereof and (b) a waxy matrix material. In a preferred embodiment, the waxy matrix material is selected from the group consisting of carnauba wax, glyceryl behenate, castor wax, and combinations thereof.

The invention also provides a method of making a solid dosage form, the comprising: (a) hot melting a waxy material to form a melt, (b) granulating in or a pharmaceutically acceptable salt thereof with the melt to form a granulate; (c) the granulate; and (d) compressing granulate to form a matrix.

### BRIEF DESCRIPTION OF THE DRAWINGS

FIG. 1 is a graph of % active release vs. time for various core formulations having carnauba wax as the only primary waxy material as compared to GLUCOPHAGE XR®.

FIG. 2 is a graph of % active release vs. time for two of the same formulations shown in FIG. 1, GLUCOPHAGE XR®, and a core formulation having behenate.

FIG. 3 is a graph of % active release vs. time for coated, but uncured formulations having 200 mg carnauba wax per core and GLUCOPHAGE XR®.

FIG. 4 is a graph of % active release vs. time for cores having 170 mg of carnauba wax per core; such cores with 6% uncured coating; and such cores with 6% cured coating compared to GLUCOPHAGE XR®.

FIG. 5 is a graph of % active release vs. time for cores having various of magnesium stearate per core as compared to Glucophage XR®.

### DETAILED DESCRIPTION OF THE INVENTION

In a preferred embodiment, the invention provides a solid dosage form comprising metformin or a pharmaceutically acceptable salt thereof, most preferably metformin hydrochloride, in a waxy matrix. The waxy matrix is preferably prepared by hot melting a suitable wax material and using the melt to granulate the metformin material.

The wax material can be any suitable wax material. Preferably, the wax material is an amorphous wax, an anionic wax, an anionic emulsifying wax, a bleached wax, a carnauba wax, a cetyl esters wax, a beeswax, a castor wax, a cationic emulsifying wax, a cetrimide emulsifying wax, an emulsifying wax, a glyceryl behenate, a microcrystalline wax, a nonionic wax, a nonionic emulsifying wax, a paraffin, a petroleum wax, a petroleum ceresin wax, a spermaceti wax, a white wax, or a yellow wax. These and other suitable waxes are described in Kibbe, Authur H., Handbook of Pharmaceutical Excipients, 3d Ed. (2000) and Remington's Pharmaceutical Sciences, 18th Ed. (1990), and are incorporated herein by reference. Preferably, the cetyl esters wax has a molecular weight between about 470 and about 490 and is a mixture consisting primarily of esters of saturated fatty alcohols and saturated fatty acids. In a preferred embodiment, the wax material is selected from the group consisting of a carnauba wax, glyceryl behenates, castor wax, and mixtures thereof. Most preferably, the wax is carnauba wax.

The wax material can be used in any suitable amount, such as, for example, from about 16% to about 35%, preferably from about 20% to about 32%, more preferably from about 24% to about 31%, and even more preferably from about 28% to about 29% of the total weight of the matrix material. When a combination of wax is used, e.g., carnauba wax and glyceryl behenate, they can be used in any ratio. Preferred formulations include the wax material component from 100 to about 85 parts carnauba wax and from 0 to about 15 parts glyceryl behenate. In a combination of carnauba wax and castor wax, for example, compositions are preferred as having the wax component from 100 to about 85 parts carnauba wax and from 0 to about 15 parts castor wax. In another embodiment of the invention, carnauba wax, glyceryl behenate and castor wax are present. In that embodiment, the carnauba wax preferably comprises at least about 85% of the waxy material and the balance of the waxy material is made up of a combination of glyceryl behenate and castor wax, in any relative proportion.

Optionally, fatty acids and fatty acid soaps can be present in the inventive dosage form. In some cases, the fatty acids and/or fatty acid soaps can replace a portion of the wax or waxes. These optional fatty acids and fatty acid soaps are preferably those that are generally used in the pharmaceutical industry as tableting lubricants, such as, for example, solid fatty acids (generally, e.g., of 16-22 carbon atoms), and their alkaline earth metal salts thereof, particularly the magnesium and calcium salts. Preferably, the fatty acid is stearic acid The optional fatty acids and fatty acid soaps, when present, are preferably used in amounts of up to about 10% of the total weight of the matrix material, preferably from about 2.5% to about 9%, more preferably from about 2.7% to about 8.6%, still more preferably from about 3% to about 6% of the total core formulation. In a preferred embodiment, an amount of up to about 2% of the total core formulation of the optional materials is used as a blend with the melt granulate. Preferably, amounts of at least 1% are used in this fashion with the remainder being added to the waxes for melting and granulating the active agent.

In a preferred embodiment, the waxes are melted and used to granulate the active agent, the granulate is allowed to cool and is then milled to a proper size and blended with processing aids. Advantageously, the granulate is milled to an average particle size of about 75 microns to about 850 microns, preferably of about 150 microns to about 425 microns.

In a preferred embodiment, processing aids are included in the dosage form. The processing aids include, for example, hydrophobic colloidal silicon dioxide (such as CAB-O-SIL®M5). Preferably, the hydrophobic silicon dioxide is not used in amounts greater than about 0.5%, but individual formulations can as required.

The blend of the waxy granulate and the processing aids, if any, is preferably compressed and then optionally coated. The coating can be any suitable coating, such as, for example, a functional or a non-functional coating, or multiple functional and/or non-functional coatings. By “functional coating” is meant to include a coating which modifies the release properties of the total formulation, e.g., sustained-release coating. By “non-functional coating” is meant to include any coating that is not a functional coating, e.g., a cosmetic coating. A non-functional coating can have some impact on the release of drug due to the initial dissolution, hydration, perforation of the coating, etc., but would not be considered to be a substantial deviation from the non-coated composition.

The functional coating preferably includes a coating agent comprising (a) a non-water-permeable component, e.g., a film former, preferably an alkyl cellulose, more preferably an ethylcellulose, such as AQUACOAT® (a 30% solution available from FMC, Philadelphia, Pa.) and SURELEASE® (a 25% solution available from Colorcon, West Point, Pa.) and (b) a water-soluble component, e.g., an agent that can form channels through the ethylcellulose upon the dissolution of the soluble component.

Preferably, the water-soluble component is a low molecular weight, non-polymeric material, e.g., a hydroxyalkylcellulose, hydroxyalkyl (alkylcellulose), carboxymethylcellulose, or salts thereof. Even more preferably, the water-soluable component is hydroxyethylcellulose, hydroxypropylcellulose, hydroxyethylmethycellulose, hydroxypropylmethylcellulose, carboxymethylcellulose, or sodium carboxymethylcellulose. Most preferably, the water-soluble component is hydroxypropylmethylcellulose, such as OPADRY® (Colorcon, West Point, Pa.). The water-soluble component is preferably of relatively low molecular weight, preferably not greater than about 25,000, and more preferably not greater than about 21,000. Mixtures of the water-soluble components can include various grades, although small amounts of additional water-soluble materials with higher molecular weights can also be present. The total of the water soluble portion (b) and non-water permeable potion (a) are preferably present in weight ratios (b):(a) of about 1:4 to about 2:1, more preferably about 1:2 to about 1:1, and most preferably about 2:3. A preferred coating solution includes SURELEASE® (available from Colorcon) as the non-water permeable portion and OPADRY® (available from Colorcon) as the water-soluble portion. In another preferred embodiment, the coating solution includes OPADRY® (Colorcon) and SURELEASE® (Colorcon) in a weight ratio of the solids content of OPADRY® to the solids content of SURELEASE® of about 2:8 to about 2:1, preferably about 2:4 to about 2:2, more preferably about 2:3, with the total coating preferably about 5% to about 6%, more preferably about 5.4% to about 5.7%, and most preferably about 5.66% of the total formulation.

In another preferred embodiment, the coating solution includes OPADRY® and AQUACOAT® (FMC, Philadelphia, Pa.) in ratios of about 1:3 to about 1:1 of OPADRY®:AQUACOAT®. While the ratios disclosed herein are preferred for duplicating target release rates of presently marked dosage forms, other ratios can be used to modify the speed with which the coating permits release of the active agent.

The coated tablets are preferably allowed to cure for at least about 1-2 hours a temperature of at about 50° C. to about 60° C., more preferably of about 55° C.

In certain preferred embodiments where the waxy material consists of carnauba wax and no other waxy material is used, the matrix is preferably coated with a functional coating, which can be cured as described herein, although curing may not be required. In certain other preferred embodiments where the matrix includes glyceryl behenates and carnauba wax, the matrix can be used without a coating, but preferably have either a cosmetic coating or a functional coating depending on the precise release profile and appearance desired.

The dosage form of the invention can include, for example, (a) compressed coated or uncoated tablets, (b) compressed pellets contained in capsules, and (c) loose powder or powder filled capsules.

In addition to the embodiments where metformin is the active agent, the dosage forms of the invention can also contain other active agents useful in the treatment of diabetic conditions, such as, for example acetohexamide, sulfonylureas (chlorpropamide, tolazamide, tolbutamide, glipizide, glyburide, glimepiride), biguanide groups (phenformin, buformin), thiazolidinediones (rosiglitazone, pioglitazone), meglitinides (repaglinide, nateglinide) and combinations hereof.

### EXAMPLES

The following examples further illustrate the invention but, of course, should not be construed as in any way limiting its scope.

### Example 1

Metformin hydrochloride tablet cores of the following formulation were prepared as follows (the first table presents the formulas in % by weight, while the second table presents the same formulas in mg amounts per 500 mg metformin hydrochloride dosage form):

| | Formula 1 | Formula 2 | Formula 3 | Formula 4 | Formula 5 |
|---|---|---|---|---|---|
| Component | Weight % | Weight % | Weight % | Weight % | Weight % |
| Matrix | | | | | |
| Metformin HCl | 81.97 | 73.86 | 70.72 | 66.00 | 67.68 |
| Carnauba wax | 16.39 | 24.67 | 28.29 | 33.00 | 27.07 |
| Glyceryl Dibehenate | | | | | 4.06 |
| Processing aids | | | | | |
| Hydrophobic colloidal | | | | | 0.11 |
| silicon dioxide | | | | | |
| (Cab-O-Sil M5) | | | | | |
| Magnesium Stearate | 1.64 | 1.48 | 0.99 | 0.99 | 1.08 |
| Total Core | 100.00 | 100.01 | 100.00 | 99.99 | 100.00 |
| | Formula 1 | Formula 2 | Formula 3 | Formula 4 | Formula 5 |
| Component | Weight (mg) | Weight (mg) | Weight (mg) | Weight (mg) | Weight (mg) |
| Matrix | | | | | |
| Metformin HCl | 500 | 500 | 500 | 500 | 500 |
| Carnauba wax | 100 | 170 | 200 | 250 | 200 |
| Glyceryl Dibehenate | | | | | 30 |
| Processing aids | | | | | |
| Hydrophobic colloidal | | | | | 0.8 |
| silicon dioxide | | | | | |
| (Cab-O-Sil M5) | | | | | |
| Magnesium Stearate | 10 | 10 | 7 | 7.5 | 8 |
| Total Core | 610 | 680 | 707 | 757.5 | 738.8 |

The metformin hydrochloride was mixed with the carnauba wax and hot melt granulated. The granulate was then milled and the magnesium stearate and colloidal silicon dioxide processing aids were added and blended. The blend was then compressed. The tablets so prepared were tested in a pH 6.8 buffer dissolution media in a side-by-side test alongside commercially available GLUCOPHAGE® (Bristol Myers Squibb) of the same strength.

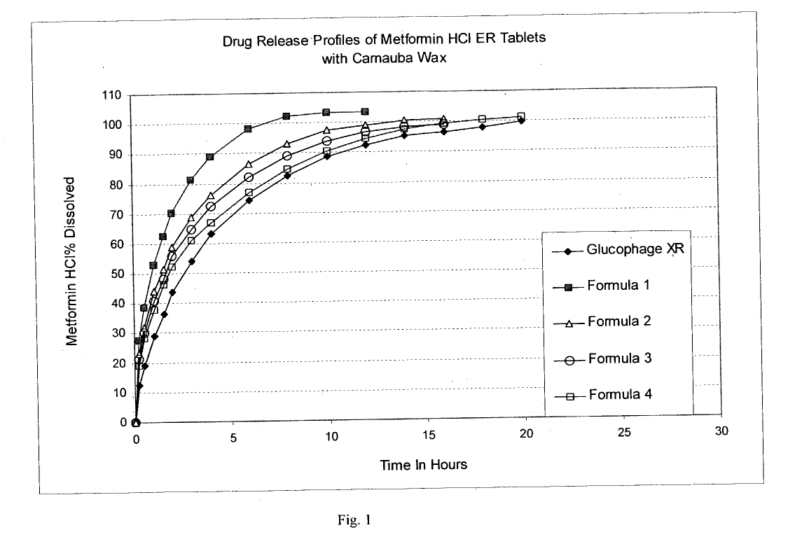

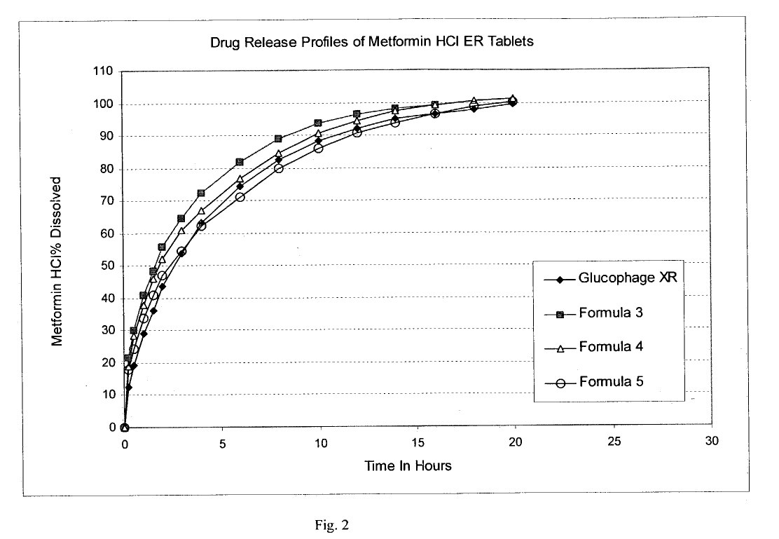

The release characteristics in pH 6.8 buffer for formulae 1-4 are shown in FIG. 1 along with that of commercially available GLUCOPHAGE® 500 mg tablets. The release characteristics of formulae 3-5 and GLUCOPHAGE XR® are shown in FIG. 2.

### Example 2

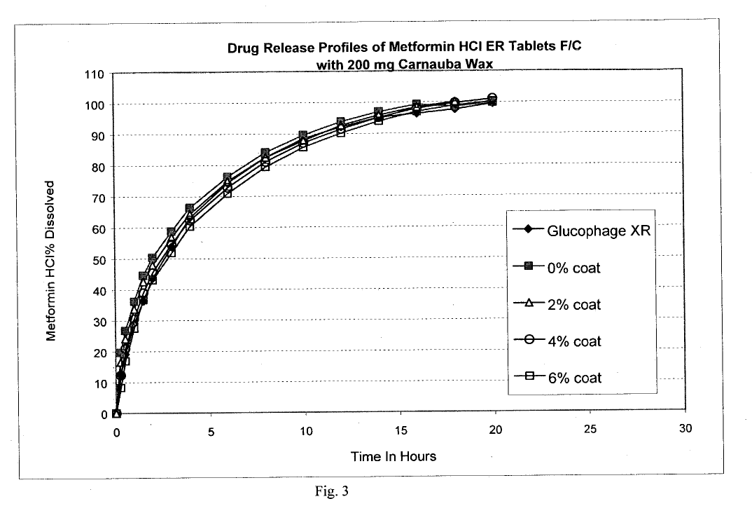

Cores of Formula 3 in Example 1 above were coated at 35° C. to 40° C. with a functional coating solution comprising 5% OPADRY®II Yellow (Colorcon), 20% SURELEASE® (Colorcon), and 75% deionized water. The coating was applied so that the coating was 2% 4%, or 6% of the total formulation. The release characteristics are shown in FIG. 3 along with uncoated cores and Glucophage XR.

### Example 3

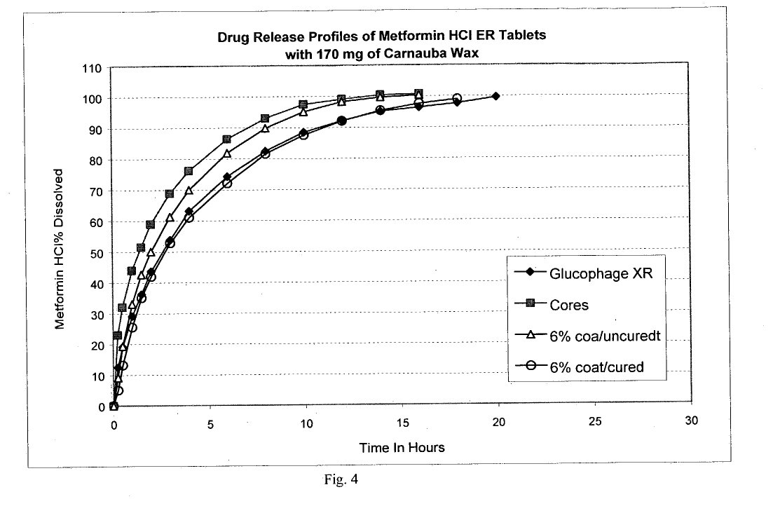

Cores of Formula 2 above (approximately 170 mg of carnauba wax/core) were coated with a functional coating solution comprising 5% OPADRY® II Yellow (Colorcon), 20% SURELEASE® (Colorcon), and 75% deionized water as in Example 2. The coating was applied so that the coating is 6% of the final formulation. A portion of the coated cores was cured for 1 hour at 55° C. The coated and cured cores were compared for release characteristics with the uncured coat, the uncoated cores and GLUCOPHAGE XR®. The results are shown in FIG. 4.

### Example 4

Cores having the following formulation were prepared and tested for % active release vs. time.

| Component | mg/core (%) | mg/core | mg/core |
|---|---|---|---|
| Metformin hydrochloride | 500(70.72) | 500(69.34) | 500(67.39) |
| Carnauba wax | 200(28.29) | 200(27.74) | 200(26.95) |
| Mg Stearate | 7(0.99) | 21(2.91) | 42(5.66) |
| | (100.00) | (100.00) | (100.00) |

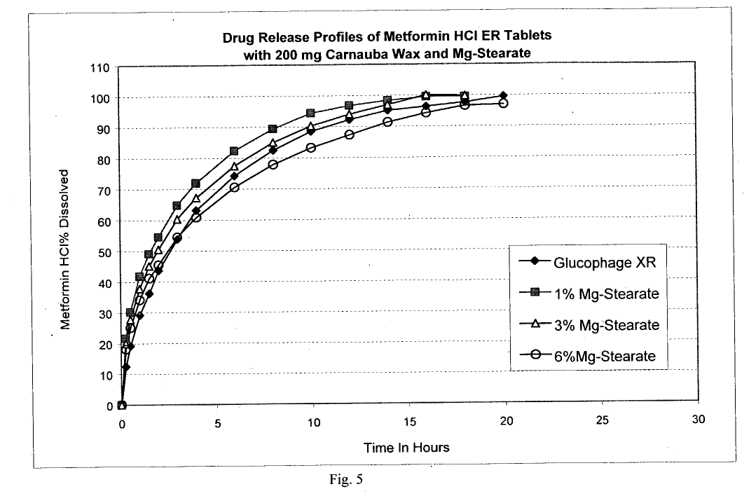

The results are shown in FIG. 5 and compared to GLUCOPHAGE XR®.

### Example 5

A further example of the invention includes the formulation set forth below.

| Matrix | % total formulation | mg |
|---|---|---|
| Metformin HCl | 69.58 | 500 |
| Carnauba wax | 23.66 | 170 |
| Processing aids | | |
| Hydrophobic colloidal | | |
| silicon dioxide | | |
| (Cab-O-Sil M5) | 0.14 | 1 |
| Magnesium Stearate | 0.97 | 7 |
| Total Core | 94.35 | |
| Coating | | |
| Opadry clear | 2.26 | 16.2 |
| Surelease Coat | 3.39 | 24.3 |
| Total coating | 5.65 | |
| Total Tablet | 100.00 | |

The metformin hydrochloride was mixed with the carnauba wax and hot melt granulated. The granulate was then milled and the magnesium stearate and colloidal silicon dioxide processing aids were added and blended. The blend was then compressed. The compressed (as yet uncoated) tablets were then coated with a blend of the coating ingredients at 35° C. to 40° C. and the tablets were cured for 1 to 2 hours at 55° C.

The tablets so prepared can be tested in a pH 6.8 buffer dissolution media in a side-by-side test alongside commercially available GLUCOPHAGE® (Bristol Myers Squibb) of the same strength. Tablets of the invention were tested when freshly prepared and after storage at 40° C. for 1, 2 or 3 months.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A solid dosage form comprising a matrix, wherein the matrix comprises:
(a) a pharmaceutically effective amount of metformin or a pharmaceutically acceptable salt thereof, and
(b) a wax material.

2. The solid dosage form of claim 1, wherein the wax material includes a material selected from the group consisting of carnauba wax, glyceryl behenate, castor wax, and combinations thereof.

3. The solid dosage form of claim 2, wherein the wax material includes carnauba wax.

4. The solid dosage form of claim 2, wherein the wax material includes glyceryl behenate.

5. The solid dosage form of claim 1, wherein the matrix is coated with a coating composition.

6. The solid dosage form of claim 5, wherein the coating composition is a functional coating composition.

7. The solid dosage form of claim 6, wherein the functional coating composition comprises:
(a) a non-water permeable component; and
(b) a water-soluble component.

8. The solid dosage form of claim 7, wherein the non-water permeable component is ethylcellulose.

9. The solid dosage form of claim 7, wherein the water-soluble component is hydroxypropylmethylcellulose.

10. The solid dosage form of claim 7, wherein the functional coating composition has a ratio of the non-water-permeable component to the water-soluble component of about 3:2.

11. The solid dosage form of claim 6, wherein the functional coating composition is present in an amount of about 5% by weight of the total composition.

12. The solid dosage form of claim 6, wherein the functional coating composition comprises a pore forming agent.

13. The solid dosage form of claim 6, wherein the functional coating composition comprises dyes, pigments, or mixtures thereof.

14. The solid dosage form of claim 5, wherein the coating composition is a non-functional coating composition.

15. The solid dosage form of claim 14, wherein the non-functional coating composition comprises a water-soluble component in the substantial absence of a non-water-permeable component.

16. The solid dosage form of claim 14, wherein the non-functional coating composition comprises pharmaceutically acceptable dyes, pigments, or mixtures thereof.

17. The solid dosage form of claim 1, wherein the matrix further comprises a processing aid.

18. The solid dosage form of claim 17, wherein the processing aid comprises a hydrophobic colloidal silicon dioxide and a member selected from the group consisting of fatty acids and fatty acid soaps.

19. The solid dosage form of claim 17, wherein the processing aid comprises a hydrophobic silicon dioxide and magnesium stearate.

20. The solid dosage form of claim 1, wherein the matrix further comprises an additional active agent.

21. The solid dosage form of claim 20, wherein the additional active agent is an active agent suitable for the treatment of diabetes.

22. The solid dosage form of claim 20, wherein the additional active agent is selected from the group consisting of acetohexamide, sulfonylureas, biguanides other than metformin, thiazolidinediones and meglitinides.

23. The solid dosage form of claim 20, wherein the additional active agent is selected from the group consisting of glyburide, acetohexamide, chlorpropamide, tolazamide, tolbutamide, glipizide, glimepiride, phenformin, buformin, rosiglitazone, pioglitazone, repaglinide, and nateglinide.

24. The solid dosage form of claim 1, wherein the matrix further comprises an additional high-dose active agent.

25. A method of making a solid dosage form comprising a matrix, the method comprising: (a) hot melting a waxy material to form a melt, (b) granulating metformin or a pharmaceutically acceptable salt thereof with the melt to form a granulate; (c) milling the granulate; and (d) compressing granulate to form a matrix.

26. The method according to claim 25, further comprising prior to step (d) blending the granulate with a processing aid.

27. The method according to claim 25, further comprising coating the matrix with a functional or a non-functional coating.

28. The solid dosage form resulting from the method of claim 25.

29. The solid dosage form of claim 1, having a size which is substantially smaller than the size of a same strength dosage form of GLUCOPHAGE® or GLUCOPHAGE XR®.

30. A tablet comprising the solid dosage form of claim 1.

31. The tablet of claim 30, further comprising a functional or non-functional coating.

32. A capsule comprising the solid dosage form of claim 1.
